(19) United States
(12) Patent Application Publication
(10) Pub. No.: US 2016/0271054 A1
(43) Pub. Date: Sep. 22, 2016

(54) Micronutrient Delivery Methods and Devices

(71) Applicants: Henry Eisenson, San Diego, CA (US); Eric Eisenson, San Diego, CA (US)

(72) Inventors: Henry Eisenson, San Diego, CA (US); Eric Eisenson, San Diego, CA (US)

(21) Appl. No.: 14/659,533

(22) Filed: Mar. 16, 2015

(51) Int. Cl. A61K 9/00; A61K 33/32; A61K 33/30; A61K 33/26; A44C 9/00; A61K 9/70; A44C 5/00; A44C 15/00; A44C 7/00; A61K 33/34; A61K 33/24

(52) U.S. Cl. CPC A61K 9/0014; A61K 33/34; A61K 33/32; A61K 33/30; A61K 33/26; A61K 33/24; A61K 9/7023; A44C 5/0023; A44C 15/005; A44C 7/00; A44C 15/0085; A44C 9/0053

(57) ABSTRACT

Metal micronutrient delivery means comprising, integrated into, plated upon, or attachable to jewelry, clothing, and personal accessories, such that they are in contact with the wearer's skin enabling micronutrients to be transferred to and potentially through the wearer's skin.

### CROSS-REFERENCE TO RELATED APPLICATIONS

Provisional Application Ser. No. 62/018,031, filed on Jun. 27, 2014.

### FEDERALLY SPONSORED RESEARCH OR DEVELOPMENT

Not applicable

### BACKGROUND OF THE INVENTION

1. Field

The present invention is in the field of personal accessories such as jewelry, with the added incorporation of micronutrients.

2. Description of the Problem

In the prior art there are numerous non-medical devices and methods claimed to be beneficial to “wellness”, including many that place magnets in contact with various parts of the human body. There is no proven benefit from the use of such items, except that users respond that they feel better, resulting from the placebo effect, which is well known in science. While magnets may provide a placebo effect, they do not have the ability to meet real medical or nutritional needs. There are no deficiencies related to magnetism that can cause medical or nutritional consequences.

The human biochemical system, like the systems of other animals and plants, depends upon a diet that provides energy and tissue building materials called “macronutrients”, plus several dietary ingredients that are required in very small quantities and therefore are called “micronutrients”. Though the generally accepted Minimum Daily Requirement for micronutrients in adults is measured in milligrams or micrograms, deficiencies in these substances can cause significant medical problems. Some micronutrients are vitamins, which are essential to general health and help avoid specific and sometimes deadly medical problems. Some micronutrients are minerals, which are also critically important to the human metabolism. Some of those critically important mineral micronutrients are metals. Examples of micronutrient metals and the result of deficiencies are set forth in Table 1 below.

TABLE 1
Example of Metal Micronutrients

| Micronutrient | |
|---|---|
| Metal | Deficiency Outcome |
| Copper | Associated with such symptoms as anemia, |
| | neutropenia, bone abnormalities, pigmentation |
| | problems, impaired growth, reduced infection |
| | resistance, osteoporosis, hyperthyroidism, and |
| | metabolic abnormalities. |
| Iron | Associated with negative affects at two or more |
| | different levels of metabolism. Macronutrient quantities |
| | of iron become a part of hemoglobin, a protein that |
| | carries oxygen throughout the body, and a gross |
| | deficiency thereof is called “anemia”. But iron in |
| | micronutrient quantities is also important to the function |
| | of certain tissues and metabolic mechanisms; as |
| | examples, iron deficiency has been shown to negatively |
| | affect memory and/or motor functions |
| Chromium | Associated with metabolic disorders because it is |
| | required for the metabolism of sugar and lipids. |
| Zinc | Associated with malabsorption of other nutrients, |
| | acrodermatitis, enteropathica, chronic liver disease, |
| | chronic renal disease, and other chronic illnesses. |
| Nickel | Associated with depressed growth rates, reproductive |
| | system changes, and lipid and glucose problems. |
| Magnesium | Associated with negative effect upon structural |
| | development of bone, the synthesis of DNA, RNA, and |
| | the antioxidant glutathione, and can suppress active |
| | transport of calcium and potassium ions across cell |
| | membranes, which is important to nerve impulse |
| | conduction, muscle contraction, and normal heart |
| | function. |
| Molybdenum | Associated with increased rates of esophageal cancer, |
| | and poor detoxification of the liver. |

These and other micronutrient problems have been generally uncommon because a normal western diet includes an adequate supply of such micronutrients, usually from plant sources. That is also true for common vitamins; deficiencies have been rare among those with a normal western diet. However, modern western society increasingly adopts highly processed food that can be rapidly prepared and consumed, and research has shown that as the human diet shifts to such foods the possibility of deficiencies grows.

3. Toxicity

While vitamin pills and other dietary supplements usually provide the common vitamins required to avoid most deficiencies caused by poor dietary habits, many such products fail to meet the need for metallic micronutrients.

Some micronutrient metals become toxic when ingested in large quantities, and some misinformed consumers gulp large quantities of vitamin pills daily, so that toxicity is one of the reasons why those who formulate typical “vitamin pills” tend to avoid the use of potentially toxic metal content. Some consumers ingest hundreds of times the Recommended Daily Allowance of certain vitamins such as Vitamin C, even against the advice of the product label and their physicians. Fortunately, Vitamin C is water-soluble and does not build up in the body. Unfortunately, vitamins such as Vitamin A are fat-soluble and can build up in the body to potentially toxic levels.

There is no practical way to provide ingestible metals as micronutrients while eliminating the risk of toxicity among consumers who believe that “more is better” and “still more is better yet.” Most micronutrient metals can be toxic in large quantities.

For each micronutrient (whether a vitamin or mineral, including metals) there is a daily intake range that satisfies the body's requirement, and a point at which toxicity occurs. Between those two limits is a range that for most people is acceptable, potentially useful, and safe. However, it is well-established that deficiencies exist, and the curve defining dosage vs. deficiency is such that in some human subjects very small quantities of additional micronutrients, including metals, can have a significant health benefit.

4. Placebo Effect

There exist many jewelry products that contain magnets, and are sold as “beneficial to health”. There is no credible evidence that such “wellness” products offer any health benefit. There has never been reproducible research showing correlation between magnet-bearing jewelry/clothing/mattresses, etc. and health, yet consumers buy such products and report feeling better—apparently due to some derivative of the ‘Placebo Effect”. Even more significant, there is no known medical condition resulting from the absence of the static magnetic fields generated by such products. On the other hand, deficiencies in metal micronutrients have predictable, quantifiable, and diagnosable medical results. This comparison differentiates the present invention from personal accessories that incorporate magnets.

It is no surprise to behaviorists that the Placebo Effect functions even when magnet-bearing jewelry is worn by people with technical backgrounds. Most people who expect a result, achieve it. It appears that humans aggressively seek reasons to feel better, to perform better. That is so even when science and common sense converge on the impossibility of a device or remedy producing positive results.

5. Objectives

One objective of the present invention is to provide users with “health-promoting” personal accessories, including jewelry, that both exploit the Placebo Effect and potentially provide a real and tangible health benefit.

Another objective is to provide physiologically safe means for delivery of metal micronutrients at a safe delivery rate.

Another objective is to provide economically viable products that accomplish said deliveries.

Another objective is to provide a means by which small quantities of micronutrient metals can be transferred to tissues with a reduced probability of toxicity.

Another objective is to provide means by which micronutrient metal payloads can be modified in response to adverse reactions.

### BRIEF SUMMARY OF THE INVENTION

The present invention provides jewelry and other accessory items that ordinarily are in contact with the skin, that contain, are made of, or have a surface providing a payload of selected micronutrients, each in a form that can permit transfer of said micronutrients to the skin tissues.

### BRIEF DESCRIPTION OF THE SEVERAL VIEWS OF THE DRAWINGS

FIG. 1 shows the general relationship between dosage of a micronutrient and deficiency, normal function, and toxicity. Values/quantities vary with the substance, but the principle remains valid.

FIG. 2 shows the process by which micronutrient metals are processed into jewelry.

FIG. 3 shows a bracelet with an additional micronutrient delivery feature.

FIG. 4 shows a disposable patch with one side containing a micronutrient delivery means and the other an adhesive, suitable for affixing to the skin-contact side of a wristwatch or other jewelry item.

FIG. 5 shows a metal accessory with one side providing a micronutrient delivery means, with bendable tabs suitable for attaching said accessory to the band of a watch or other jewelry item.

FIG. 6 shows examples of common jewelry made of or providing micronutrient delivery means.

FIG. 7A shows an example of a stainless steel bracelet with a metal strip configuration.

FIG. 7B shows an example of a stainless steel bracelet configured to provide other metals as rivets or screwed-on devices.

FIG. 7C shows an example of a bracelet configured to provide multiple metal micronutrients, as rivets.

FIG. 8 shows the use of payload patches in hats, socks, wristbands, headbands, and footwear.

FIG. 9 shows clothing made of micronutrients expressed as wires or threads

### DETAILED DESCRIPTION

There exist many jewelry products in the marketplace that contain magnets, and are sold as “beneficial to health”. There is no credible evidence that such products offer any health benefit. There has never been reproducible research showing correlation between magnet-bearing jewelry/clothing/mattresses, etc. and health, yet consumers buy such products and report feeling better—apparently due to some derivative of the ‘Placebo Effect”. It is no surprise to behaviorists that the Placebo Effect functions even when magnet-bearing jewelry is worn by people with technical backgrounds. Most people who expect a result achieve it. It appears that humans aggressively seek reasons to feel better, to perform better. That is so even when science and common sense converge upon the impossibility of positive results from some device, food supplement, or remedy.

The instant invention is not only a stimulant of the Placebo Effect, but works on the probability that when micronutrient metals come in contact with the skin there can be a finite transdermal absorption of micronutrient metals in the form of elemental metals, metal oxides, or metal salts. For example topical magnesium is absorbed through the skin, as is the payload of many medical patches. In fact, many substances do pass into the body from the outer surface of the skin into the circulation. To understand how this works, imagine a tightly woven fabric. While from a distance it may appear impervious, at close range it is actually highly porous. It is this porous nature of the skin, with its millions of tiny openings, that allows not only sweat and toxins to escape, but also enables the absorption of some substances. That absorption is facilitated by any fluid, including perspiration.

The process is known as dermal absorption. Once a substance passes through the outer layers of skin, it passes into the lymph and local vascular (blood vessel) system and soon thereafter into the bloodstream. While the exact mechanisms of skin transfer are not completely understood, three routes of penetration have been hypothesized:

Intercellular Skin Absorption, which occurs between the cells of the “stratum corneum”, the outermost layer of the skin;

Transcellular Skin Absorption, where substances actually pass through the skin cells themselves; and

Skin Absorption Through the Follicles and Glands, also known as “appendageal absorption”, which may also exhibit “reservoir effects” in which substances may be stored within glands for absorption over time.

Skin Permeability: The Good and The Bad

Some of the most convincing stories of substances passing into the body via the skin come from governmental agencies actively studying and monitoring dermal absorption through their chemical safety divisions.

A 2005 report published by the World Health Organization takes a very clear position on skin permeability:

“While the skin does act as a barrier, it is not a complete barrier. Many chemicals do penetrate the skin, either intentionally or unintentionally, and cutaneous metabolism does occur. Because of its large surface area, the skin may be a major route of entry into the body . . . .”

This “major route of entry” has become a concern in many circumstances where toxic substances are released into air, water, and even city water supplies.

The California Environmental Protection Agency issued a report entitled “Chlorinated Chemicals in Your Home”, warning of the risks of cancer due to chlorinated chemicals. The agency issued the statement: “Taking a long, hot shower in a typical small shower stall can substantially increase your exposure to chloroform. If you use indoor spas, hot tubs, or swimming pools, you are also likely to be exposed to high levels of chloroform.”

Health Canada has estimated that skin exposure to certain toxic hydrocarbons in the Great Lakes may be as dangerous as oral exposure, issuing alerts to bathers, especially those affected by sunburn, which may enhance absorption.

Worker safety is an issue. Workers in various industries have suffered poisoning, in some cases fatal, from substances penetrating exclusively through the skin and into the bloodstream, such as through dermal exposure to leaded gasoline and insecticides.

The European Commission and the World Health Organization have both issued Guidance Documents, such as the “Guidance Document on Dermal Absorption” and International Programme on Chemical Safety Environmental Health Criteria serve to instruct agencies on how to protect workers from exposure to toxic compounds. The absorption of metals through the skin has been shown and considered to be occurring, as noted in the health Risk Assessment Guidance for Metals, Fact Sheet 01 published in August of 2007. The instant invention here provides for the contact with the skin and perspiration to provide exposure to the skin for transdermal absorption of the key metal micronutrients of Table 1. While such government agencies work to stop the transfer of chemicals through the skin, transdermal drug delivery products seek to take advantage of it. Transdermal patches are produced as delivery systems for nicotine, hormones, painkillers, and other substances.

These methods often provide clear advantages over oral medications, as outlined by Stanley Scheindlin, pharmaceutical chemist, in the journal Molecular Interventions:

“Patients often forget to take their medicine, and even the most faithfully compliant get tired of swallowing pills, especially if they must take several each day. Additionally, bypassing the gastrointestinal (GI) tract would obviate the GI irritation that frequently occurs and avoid partial first-pass inactivation by the liver.”

This instant invention looks at this dermal absorption effect to provide micronutrients from alloys containing the critical micronutrients of Iron, Zinc, Chromium, Manganese, Copper, Molybdenum, and Nickel, or any other metal micronutrients, that come in contact with the skin in the form of base metal, metal oxide or metal salts. The dermal absorption may occur with the alloys in contact with the skin. The alloys could incorporate all of the micronutrient metals such as Iron, Zinc, Chromium, Manganese, Copper, Molybdenum, and Nickel.

Depending on the alloy, a combination of the above metals would incorporate all, or any combination of, the metal micronutrients offered by the instant invention. Theoretical combinations of alloys with the constituent micronutrient metals contained therein could be almost infinite in number, but the alloys must be workable in a way that can be made into jewelry and other wearable items or accessories and at a reasonable cost to provide for a commercial product that can be commercially available for sale and use. That all being noted, the alloys will most likely be those that are principally of iron with the remaining constituents, Zinc, Chromium, Manganese, Copper, Molybdenum, and Nickel alloyed with the Iron. Table 2 below sets forth the range of constituents that could be expected for an Iron based alloy.

TABLE 2
Iron Alloying Ranges

| Metal | Percentage |
|---|---|
| Iron | 50% to 99% |
| Zinc | 0.1% to 2% |
| Chromium | 0.1% to 18% |
| Manganese | 0.1% to 2% |
| Copper | 0.1% to 2% |
| Molybdenum | 0.1% to 2% |
| Nickel | 0.1% to 14% |

Table 3 below sets forth the range of constituents that could be expected for copper based alloys.

| Metal | Percentage |
|---|---|
| Copper | 50% to 99% |
| Iron | 0.1% to 2% |
| Zinc | 0.1% to 50% |
| Chromium | 0.1% to 2% |
| Manganese | 0.1% to 2% |
| Molybdenum | 0.1% to 2% |
| Nickel | 0.1% to 2% |

It is important that the alloy contain combinations of Iron, Zinc, Chromium, Manganese, Copper, Molybdenum, and Nickel. They can be in varying percentages for example, Iron could be 99.4% and each of the remaining micronutrients can be each 0.1%. This can hold for each micronutrient element. Zinc could be 99.4% and each of the remaining micronutrients can be 0.1%. Chromium could be 99.4 and each of the remaining micronutrients can be 0.1%. Manganese could be 99.4% and each of the remaining micronutrients can be 0.1%. Copper could be 99.4% and each of the remaining micronutrients can be 0.1%. Molybdenum could be 99.4% and each of the remaining micronutrients can be 0.1%. Nickel could be 99.4% and each of the remaining micronutrients can be 0.1%.

There are numerous combinations of acceptable percentages, as long as the alloy contains measurable amounts of Iron, Zinc, Chromium, Manganese, Copper, Molybdenum, and Nickel, and any other metal micronutrient deemed useful, but commercial viability supports the percentage ranges in the two Tables for the Iron based alloys and the Copper based alloys, which would be more readily used, and the invention herein provides for the creation of alloys that contain the key micronutrients noted. The examples in the preceding paragraph can be accomplished and are incorporated as alloys, even though they may not have been purely commercially chosen they could still be used for many other reasons, including aesthetic appearance.

The key aspect is to have the elemental metals of Iron, Zinc, Chromium, Manganese, Copper, Molybdenum, and Nickel as the micronutrient payload placed in contact with the skin. The payload can be in the form of the alloys above or they can take other forms, or be part of other compounds, as required to place the payload proximate to the user's skin.

All drawings are for the purpose of describing examples of versions of the present invention and are not intended to limit its scope. The present invention encompasses all products, and particularly jewelry products, designed to place a micronutrient payload in contact with the skin.

The present invention provides for jewelry configurations in which sections of the jewelry contain areas in which metal micronutrients are present as elemental metals, or the salts/oxides of such metals, in alloys that enable perspiration to become a transfer medium, thus permitting passage of the micronutrients into the skin. Such micronutrients comprise the “payload” of the jewelry item.

In one embodiment of the present invention, the payload can be provided as a metallic alloy that combines the selected micronutrients in a ratio defined by a combination of:

a. Ability of a particular metallic micronutrient, in elemental form or as a salt or oxide, to pass transcutaneously into tissues.

b. Probability of deficiencies in a typical user.

c. Generally accepted Minimum Daily Requirement (MDR) for each micronutrient.

d. Identified sensitivities by prospective user(s).

In this embodiment of the present invention, the micronutrient payload is provided as a permanent part of the jewelry item. In linked jewelry, links can each be made from one of the selected payload micronutrients. In jewelry consisting of a solid band, as in a bracelet, the metal can be a homogeneous combination or alloy, comprised of various combinations of the metal micronutrients selected for the payload. Stranded jewelry can be made from wires, each comprised of one or more of the selected micronutrient payload, twisted and bonded to form the visual impression of a cable.

In another embodiment, the payload is carried in an add-on accessory that can be fitted onto existing jewelry. Said accessory can be a band around the original jewelry piece, or can be of any other form factor that will be supported by the original jewelry piece and will permit contact between the payload and skin.

In a further embodiment, the payload is a removable addition to or part of jewelry that can be replaced when the payload is depleted. One method consistent with this embodiment uses a bracelet of common metal such as stainless steel 316L, with individual micronutrients or combinations of micronutrients expressed as one or more separate band(s) that clip(s) circumferentially around the long axis of that basic bracelet. This embodiment has the additional advantage of permitting the removal of a micronutrient band to which the wearer reacts adversely.

In another embodiment, a bracelet can be manufactured with holes along its long axis, with selected individual metal micronutrients available as rivets or screws that can be mounted through those holes, placing a flattened surface against the user's skin. This embodiment has the additional advantage of enabling selection of add-ons based upon the user's needs, with the possibility that a known deficiency of one micronutrient will be compensated by using multiple rivets or screws of that material.

In another embodiment, the payload can be plated upon a metal surface, with sufficient plating thickness to provide the desired effect for the desired lifetime. Besides plating, the payload can be deposited by means of vapor deposition, plasma or thermal spraying, or ion beam techniques. In an extension of this embodiment, the payload can be micronutrients added to inks that are then printed onto the surface to be placed against the skin.

In another embodiment, the payload can be plated or printed upon one side of a substrate made of metal foil, fabric, paper or plastic, with an adhesive on the other side, so the present invention can be adhered to the back of a watch, amulet, bracelet, or other jewelry item.

In all configurations and embodiments, the payload can be comprised of individual metal bands or sections, each of which is made of one discrete micronutrient metal, such as Iron, Zinc, Chromium, Manganese, Copper, Molybdenum, and Nickel.

In all configurations and embodiments, the payload can be comprised of one alloy or multiple alloys, each of which includes multiple selected metal micronutrients.

In all configurations and embodiments, the payload can be comprised of selected metal micronutrients delivered as chemical compounds, such as a salt or oxide of selected micronutrients.

In all configurations and embodiments, individual metal micronutrients can be built into or onto the device with an exposed area proportional to the human requirement for that element, with consideration of the ability of that micronutrient to transfer transcutaneously, Recommended or Minimum Daily Requirement (RDR, MDR), and user sensitivities to specific micronutrient materials.

In all configurations and embodiments, the payload can be comprised of selected metal micronutrients, subject to sensitivities or allergies of the wearer. For example, a device, garment, or personal accessory can be so constructed that the wearer can selectively attach buttons, pads, bands, patches, or links, each providing a desired micronutrient or combination of micronutrients, thereby permitting the deletion of any micronutrient to which the user reacts adversely.

The present invention is primarily a means by which micronutrients are made available for transcutaneous delivery, and can provide mineral or vitamin micronutrients suitable for delivery by the method of placing a payload of such materials on the skin of the user. All references herein to “micronutrients” shall include micronutrient metals, minerals, and vitamins, and compounds containing them, which the present invention places in contact with the skin to enable dermal absorption.

All wrist bands, neckbands, jewelry items, accessories for jewelry, and clothing items intended to bring sources of micronutrients into contact with the skin are within the scope of the present invention, whether said payloads are components of an alloy or as separate metals, are metals or minerals, or include vitamins or other non-metallic micronutrients

In one embodiment of the present invention, the payload can be provided by a patch to be adhered to the skin, in a manner similar to that used by nicotine and pain-suppression patches.

In one embodiment of the present invention, the payload can be provided in clothing items, such as a hat in which the band provides micronutrients to the skin of the wearer, and in socks where threads include a micronutrient payload. There can be a woven section of clothing containing the micronutrients for use as socks, stocking, arm band, wristband, headbands and hats.

In a still further embodiment, there can be a replaceable patch containing the payload that can be added to clothing, hats, gloves and footwear or can be supplied with the patch already adhering to these items. The patch can be removed and replaced once there is an indication on the patch that replacement is necessary. Such an indication could be a change of color to the patch or the appearance of a message indicating the need for replacement after the outer payload is worn away.

The objectives of the present invention can be met with fabric, in which the micronutrient payloads are expressed as threads that are woven into the overall material of which clothing is made.

All means by which micronutrients are placed in contact with the user's skin, thus creating an opportunity for transcutaneous transfer, are considered to be within the scope of the present invention.

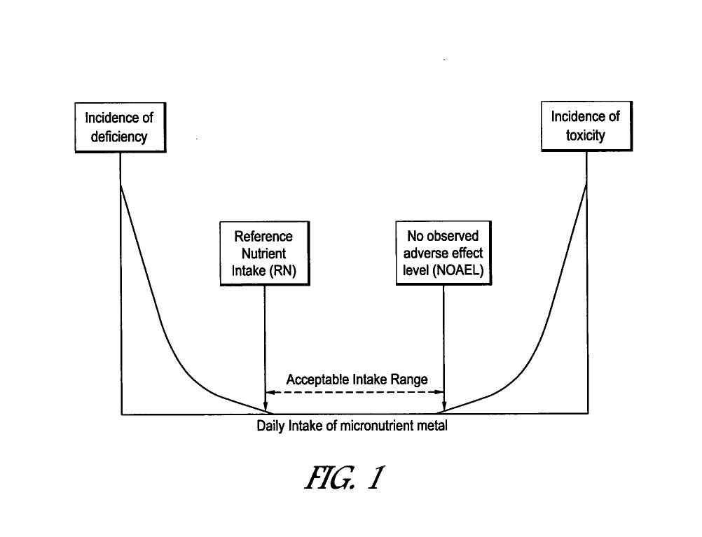

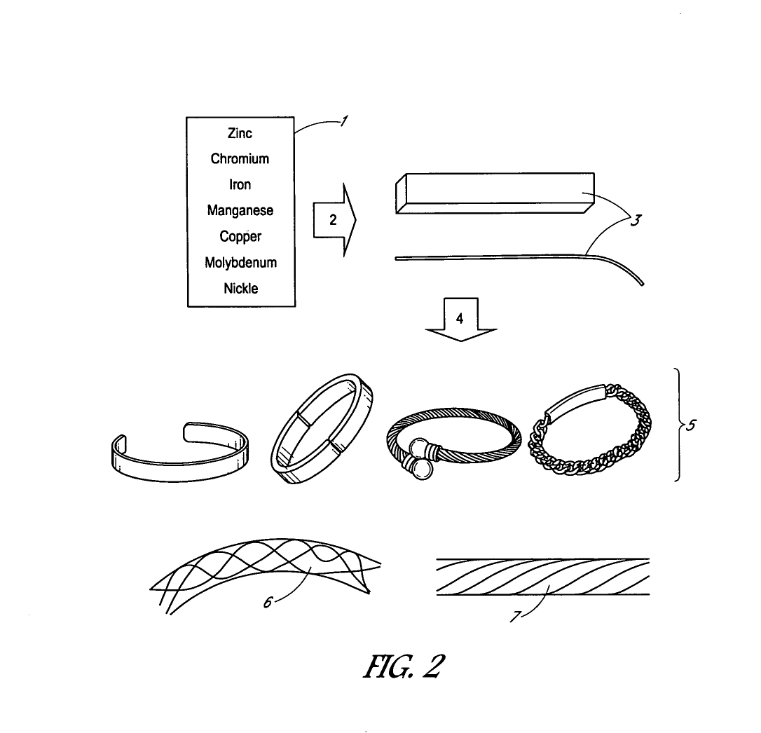

FIG. 1 shows the relationship between micronutrient intake and both deficiency and toxicity. FIG. 2 shows the process of conversion of metal micronutrients 1 via an alloying process 2 into metallic forms 3 usable for jewelry making, and then a manufacturing process 4 resulting in consumer jewelry 5. When the micronutrient-bearing metal is expressed as wires, each representing one micronutrient, they can be randomly combined in a jewelry component as 6, or twisted into a cable as 7, for further manufacturing steps. Components 6 and 7 can be wires wrapped around a base metal, plastic, leather, fabric, or a composite of materials. The wires can also be woven and made into jewelry for wearing in contact with the skin or wrapped around a base metal, plastic, leather, fabric, or a composite of materials. The metal micronutrients will contact the skin.

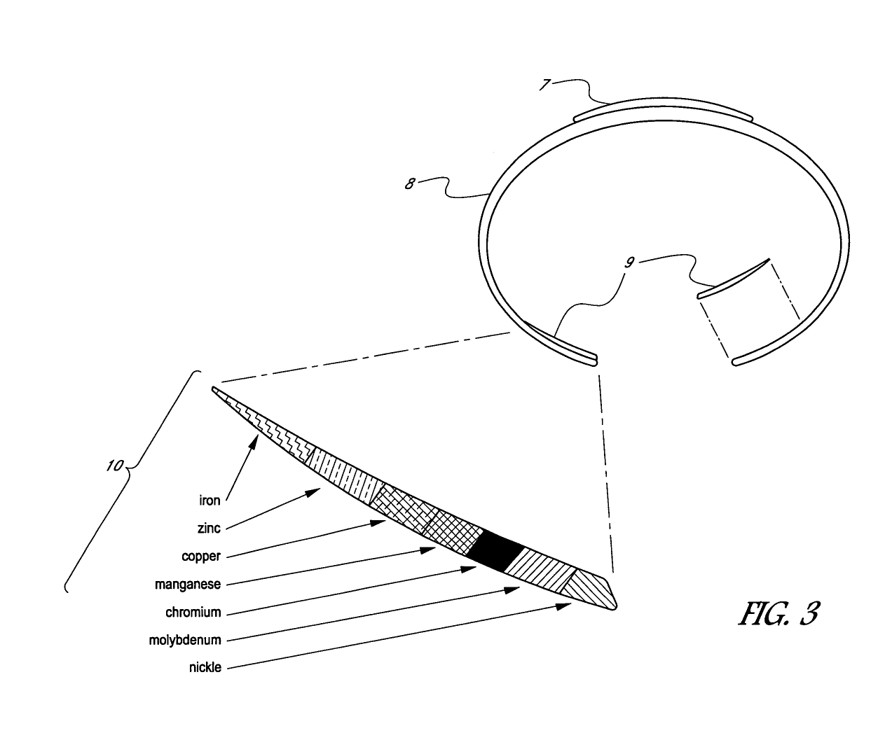

FIG. 3 shows a typical jewelry item 7 with a band 8 to which a micronutrient payload 9 has been affixed, said band comprised of either an alloy or individual micronutrient metals 10.

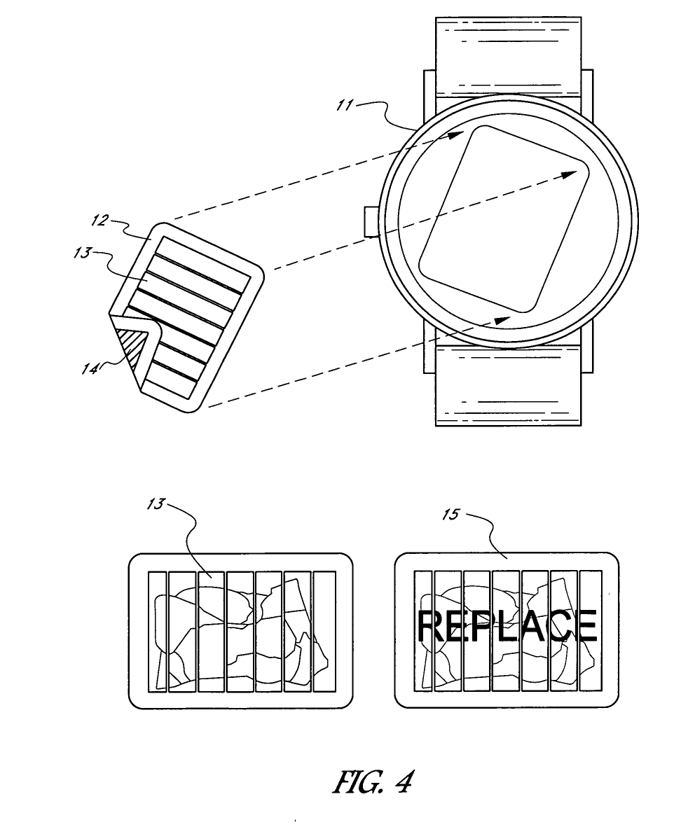

FIG. 4 shows a common wristwatch 11 to which the present invention is applied as a patch 12 with the micronutrient payload 13 on one side and an adhesive 14 on the other. The payload can be printed or plated as an alloy or with bands providing individual micronutrients, covering a REPLACE notice 15 that becomes visible after some period of wear.

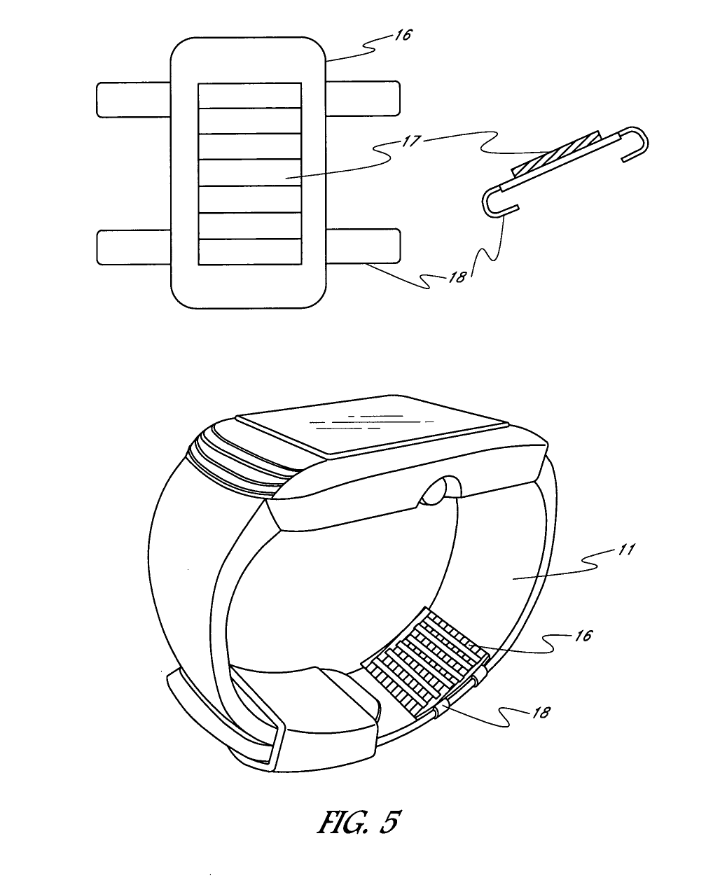

FIG. 5 shows a metal accessory 16 comprised of a micronutrient payload 17, with tabs 18 that can be bent, enabling attachment to another item such as jewelry or a wrist watch 11

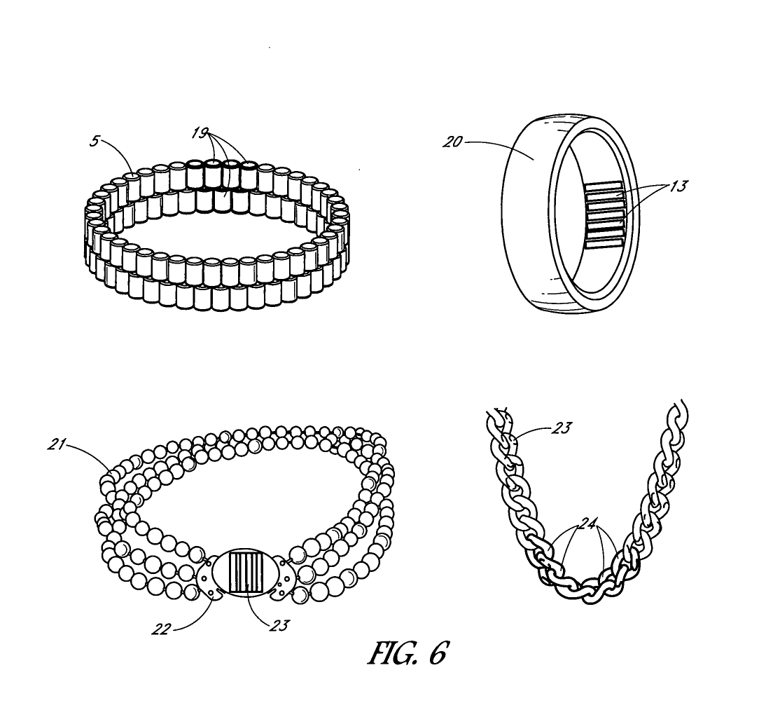

FIG. 6 shows a linked bracelet 5 of which certain links 19 are comprised of micronutrient metals, as elemental metals or alloy. Also shown is a common ring 20 with a micronutrient payload 13, a necklace 21 with clasp 22 to which a payload 13 is affixed, and a bracelet or necklace 23 with linked parts of which some 24 are fabricated from individual micronutrient metals or alloys, to deliver the payload provided by the present invention.

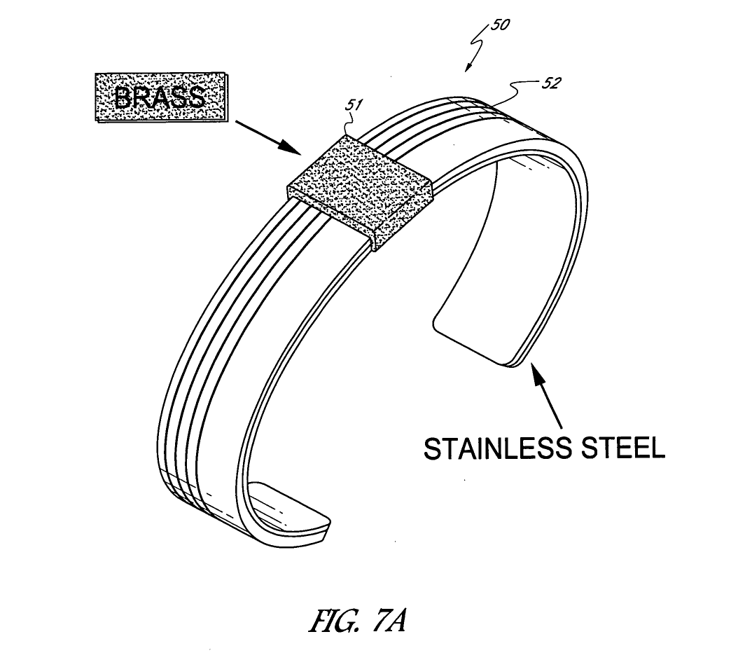

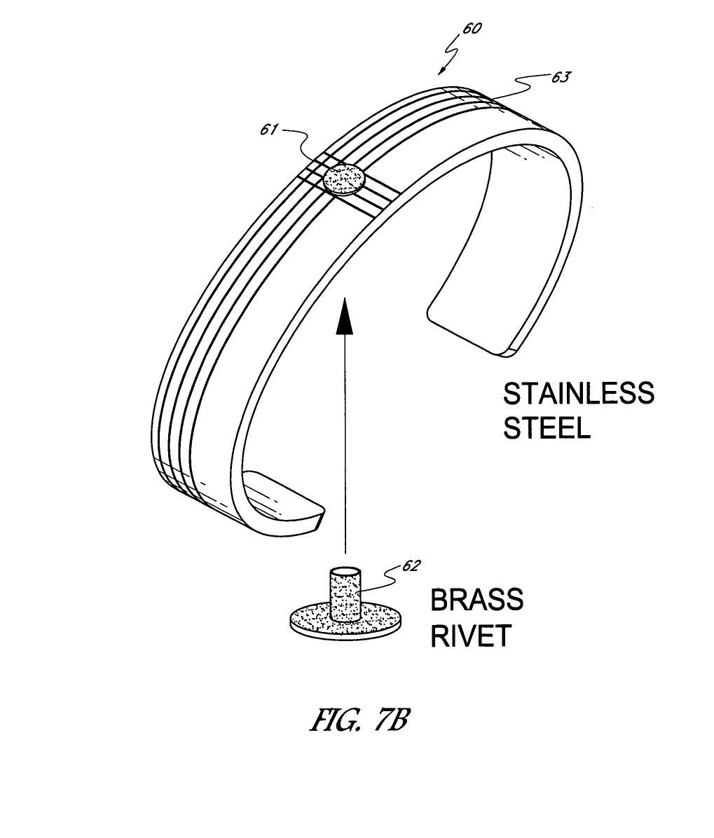

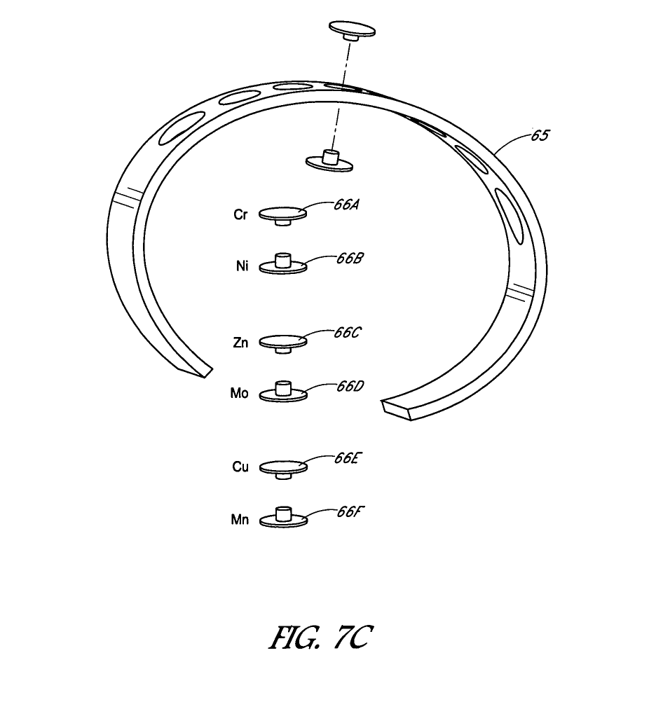

FIG. 7A and FIG. 7B shows a stainless steel bracelet. The stainless steel would be from the 300 Series because of superior corrosion resistance and body compatibility. Of the 300 Series, 316 and 316L are most commonly placed in contact with the body. Below at Table 3, is the composition of 316 and 316L. While 304 stainless is the most commonly used austenitic stainless and can be used here, the preference would be for 316 and 316L as it has superior corrosion resistance and forms as easily as 304. 316L stainless steel is preferred where there is welding because of the lower carbon content. Welding and other joining will be required for certain articles of jewelry.

TABLE 3
316 Stainless steel and 316L stainless steel

| Constituent | 316 SS % | 316L SS % |
|---|---|---|
| Carbon | 0.08 max | 0.03 max |
| Manganese | 2.00 max | 2.00 max |
| Phosphorus | 0.045 max | 0.045 max |
| Sulfur | 0.030 max | 0.030 max |
| Silicon | 0.75 max | 0.75 max |
| Chromium | 16.00-18.00 | 16.00-18.00 |
| Nickel | 10.00-14.00 | 10.00-14.00 |
| Molybdenum | 2.00-3.00 | 2.00-3.00 |
| Nitrogen | 0.10 max | 0.10 max |
| Iron | Balance | Balance |

FIG. 7A illustrates a bracelet that can be of any desired combination of metals and alloys. In this example, the bracelet 50 is manufactured from either 316 or 316L stainless steel and has an added strip of brass 51, an alloy of copper and zinc (metal micronutrients missing from 316 and 316L stainless steel). The brass addition will also comprise a potential cosmetic benefit depending on the design. Bracelet 50 has a surface 52 with shallow trenches that in alternative configuration can be strips or wires adhered in these trenches and the configuration could then be reversed with those wires or strips coming in contact with the user's skin. The strips or wires can be alloys or metals that would complete the metal micronutrients desired in the bracelet or piece of jewelry.

FIG. 7B illustrates bracelet that can be of any desired combination of metals and alloys. In this example, the bracelet 60 is manufactured from either 316 or 316L stainless steel and has an added rivet 61 and 62 of brass, an alloy of copper and zinc (metal micronutrients missing from 316 and 316L stainless steel). The brass addition will also provide an added potential cosmetic benefit depending on the design. Bracelet 60 has a surface 63 with shallow trenches that in an alternative configuration can be strips or wires adhered in these trenches and the configuration could then be reversed with those wires or strips coming in contact with the user's skin. The strips or wires can be alloys or metals that would complete the metal micronutrients desired in the bracelet or piece of jewelry.

The base metal of the bracelet can be any formable metal and added strips, rivets, wires, plating, and metal deposition can be utilized to provided the micronutrient metals needed or desired.

FIG. 7 C illustrates a bracelet 65 with rivets of various elements 66A, 66B, 66C, 66D, 66E, 66F, where the bracelet can be made of a base metal such as iron or copper and the rivets will be of the elements or alloys that will provide some or all the micronutrient metals found in Table 1. Also, the bracelet could be of a non-metal such as plastic, composite, leather, or any material capable of placing rivets containing the micronutrient metals in contact with the skin. There can be single rivets of each elements or alloy rivets containing multiple elements of the micronutrient metals.

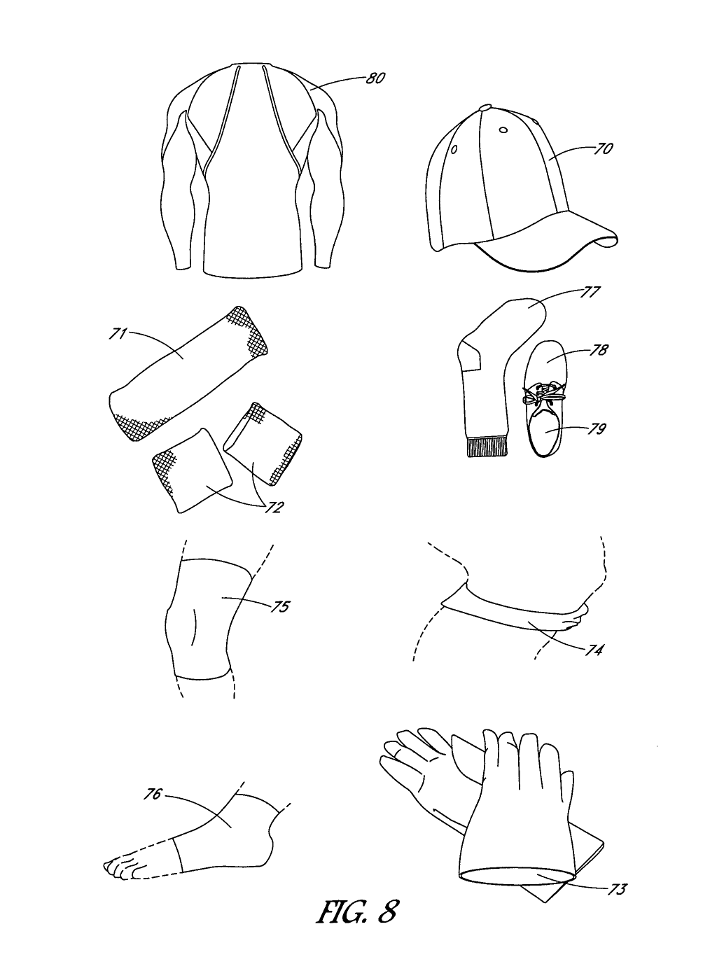

FIG. 8 shows where payload patches are replaceable and adhering to the interior of a hat 70, to a headband 71, to a wristband 72, the interior of gloves 73, a knee strap brace 74, a knee brace 75, an ankle brace 76, socks 77, shoes 78 with shoe insert 79 having the patch and workout clothes 80.

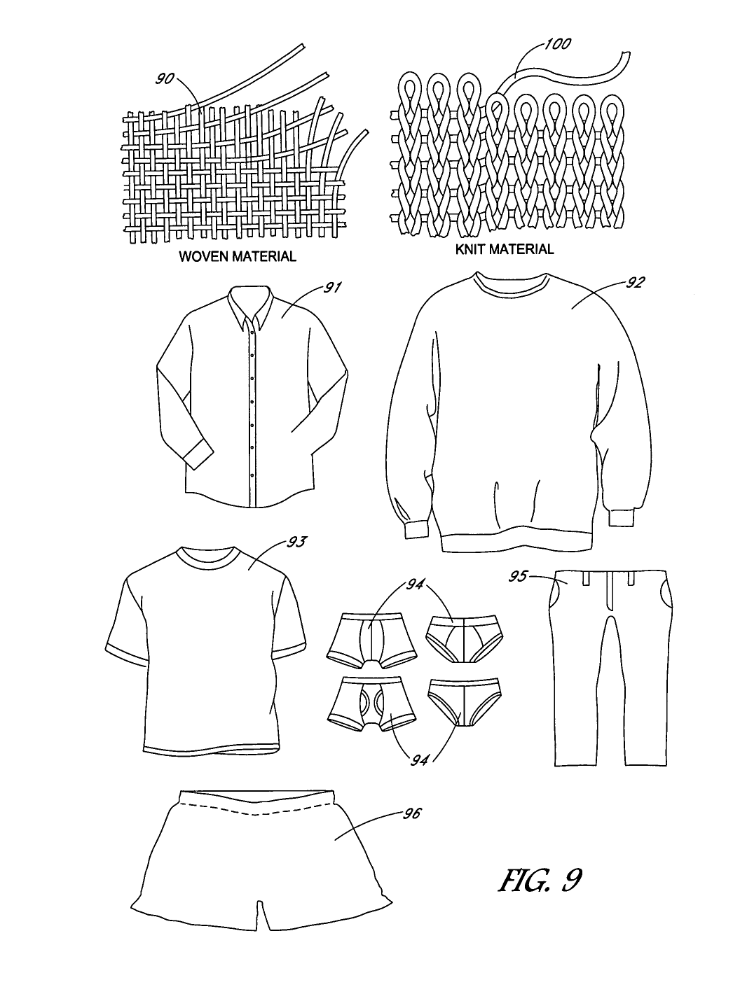

FIG. 9 shows material 90 and 100 for clothing or lining for clothing such as shirt 91, sweatshirt 92, t-shirt 93, underwear 94, pants 95 and shorts 96. The type of clothing is only limited by the imagination, as the micronutrients can be woven or knitted into fabrics used to manufacture all types of clothing.

The clothing herein can be any kind of clothing from winter wear to summer wear, the key is to either have the replaceable adhering payload patch on the clothing in contact with the skin or clothing manufactured with the micronutrient metals in the material.

The jewelry herein can be any kind of jewelry including but not limited to bracelets, necklaces, earrings, tiaras, anklets, watches, hoops for the arm and wrist, and rings. Bracelets can include those worn around wrist, ankle, leg, knee, arms, head, and neck. These bracelets can be used for sports or for any other use or just worn as an adornment.

The payload can be a patch that is applied on watches, jewelry, hats, footwear, and clothing with the patch having an indicator telling a user to change the payload patch. The patch can be on the interior or a headband or a hat. It can be in shoes, socks, pants, and under garments, where the clothing is woven with sections of the micronutrients in the form of metal wires or threads.

The payload can be any micronutrient capable of being placed in contact with the skin in a manner consistent with the methods illustrated herein.

The present invention is not limited to the embodiments described here. The rights sought are rather defined by the following claims, within the scope of which many modifications can be envisaged.

We claim:

1. Micronutrient delivery method comprising:
micronutrient metals in contact with skin permitting dermal absorption of the micronutrient metals.

2. An alloy comprising:
detectable amounts of Iron, Zinc, Chromium, Manganese, Copper, Molybdenum, and Nickel.

3. The alloy of claim 2, where Iron comprises at least 50% of said alloy.

4. The alloy of claim 2 where the Zinc, Manganese, Copper, and Molybdenum each comprises no more than 2% of said alloy.

5. The alloy of claim 2 where the Chromium comprises no more than 18% of said alloy.

6. The alloy of claim 2 where the Nickel comprises no more than 14% of said alloy.

7. A patch for delivering micronutrients comprising: where the patch is placed in contact with the skin and said patch has detectable amounts of Iron, Zinc, Chromium, Manganese, Copper, Molybdenum, and Nickel.

8. The patch of claim 7 where the detectable amount of Iron, Chromium, Zinc, Manganese, Copper, Molybdenum, and Nickel are each in the form of oxides.

9. The patch of claim 7 where the detectable amount of Iron Chromium, Zinc, Manganese, Copper, Molybdenum, and Nickel are each in the form salts.

10. The patch of claim 7 where the patch is removably attachable to jewelry.

11. The patch of claim 7 where the removably attachable patch has a surface that can wear away, exposing an indicator indicating that the patch needs to be replaced.

12. Jewelry comprising wires containing detectable amounts of Zinc, Chromium, Iron, Manganese, Copper, Molybdenum, and Nickel.

13. The jewelry of claim 12 where the wires are in contact with the skin.

14. The invention of claim 12 where the wires are woven.

15. The process where detectable amounts of Zinc, Chromium, Iron, Manganese, Copper, Molybdenum, and Nickel are deposited on the surface of material.

16. An apparatus comprising:
metals known to be micronutrient provided as elemental materials expressed as a two-dimensional array of strips of which each strip is comprised of one micronutrient metal or two or more such metals as an alloy, an assembly woven of discrete wires of which each wire is comprised of one selected micronutrient metal or two or more such metals as an alloy, or an alloy comprised of a measurable concentration of such micronutrient metals, where said strips, wires, or alloys are in the form of a bracelet, wrist watch band, or adhesive stamp, such that said micronutrient metals are held proximate to the skin of the user.

17. An alloy of Copper comprising:
detectable amounts of Iron, Zinc, Chromium, Manganese, Molybdenum, and Nickel.

18. The alloy of claim 17 comprising at least 50% of said alloy being copper.

19. The alloy of claim 17 where Zinc, Chromium, Iron, Manganese, Molybdenum, Nickel comprises are each no more than 2% of said alloy.

20. The method of delivering micronutrients comprising:
an adhering removable patch containing detectable amounts of micronutrients; and
the adhering removable patch contacting the skin providing for dermal absorption.

21. Jewelry comprising:
300 Series stainless steel and detectable amounts of copper and zinc in contact with the skin.

22. The invention of claim 21 where the detectable amounts of copper and zinc are in the form of rivets in the jewelry

23. The invention of claim 21 where the stainless steel is either 316 stainless steel or 316L stainless steel.

24. Micronutrient delivery method comprising:
micronutrient metals in contact with skin permitting dermal absorption of the micronutrient metals via the medium of perspiration.
